(19) United States
(12) Patent Application Publication
(10) Pub. No.: US 2023/0045562 A1
(43) Pub. Date: Feb. 9, 2023

(54) LIVING BODY DETECTION DEVICE, LIVING BODY DETECTION METHOD, AND PROGRAM

(71) Applicant: DATA SOLUTIONS, INC., Tokyo (JP)

(72) Inventors: Tomoaki OTSUKI, Yokohama-shi (JP); Chen YE, Yokohama-shi (JP)

(73) Assignee: DATA SOLUTIONS, INC., Tokyo (JP)

(21) Appl. No.: 17/906,561

(22) PCT Filed: Mar. 11, 2021
(86) PCT No.: PCT/JP2021/009768

§ 371 Date: Sep. 16, 2022

(30) Foreign Application Priority Data
Mar. 17, 2020 (JP) 2020-046621

(51) Int. Cl. G01S 13/56; A61B 5/113

(52) U.S. Cl. CPC G01S 13/56; A61B 5/113; G01S 7/415

(57) ABSTRACT

An object is to accurately determine the presence or absence of a living body. A living body detection device comprises: a signal acquirer that acquires a first signal including a first frequency component that is a frequency component of heartbeat and a second frequency component that is a frequency component of breathing; a filter that attenuates a frequency component higher than the first frequency component based on the first signal to generate a second signal; a frequency analyzer that analyzes a frequency component of the second signal; a variance value calculator that calculates a first variance value of energy of at least one of the first frequency component and the second frequency component based on a result of analysis by the frequency analyzer; a first statistical quantity calculator that calculates a first statistical quantity of the first variance value for a predetermined period; and a determiner that determines presence or absence of a living body based on the first statistical quantity.

### TECHNICAL FIELD

The present invention relates to a living body detection device, a living body detection method, and a program.

### BACKGROUND ART

There is a known technique in which biological information such as a heart rate is measured with a wearable device and a notification is made to a user when there is an abnormality in the biological information (see Non-Patent Literature 1, for example).

In watching systems, observation equipment such as a nurse call button, a human detection sensor, a Doppler sensor, a heart rate monitor, a breath measurement device, a thermo camera, a sphygmomanometer, a clinical thermometer, an illuminometer, a thermometer, or a hygrometer is first connected to an observed person such as an elderly person. The watching system thus acquires observation information for the observed person. The watching system then determines whether or not an emergency notification condition is met based on the observation information, and makes an emergency notification in the case of an emergency. Watching systems that use such vital sensors are known (see Patent Literature 1, for example).

### CITATION LIST

### Non Patent Literature

Non-Patent Literature 1: “Your heart rate. What it means, and where on Apple Watch (R) you'll find it.”, [online], Jan. 21, 2020, [retrieved on Mar. 2, 2020], Internet <URL: https://support.apple.com/ja-jp/HT204666>

### Patent Literature

Patent Literature 1: Japanese Patent Laid-Open No. 2017-151755

### SUMMARY OF INVENTION

### Technical Problem

In view of the fact that it has been difficult for conventional techniques to accurately determine the presence or absence of a living body, it is an object of the present invention to accurately determine the presence or absence of a living body.

### Solution to Problem

A living body detection device is required to comprise:
a signal acquirer that acquires a first signal including a first frequency component that is a frequency component of heartbeat and a second frequency component that is a frequency component of breathing;
a filter that attenuates a frequency component higher than the first frequency component based on the first signal to generate a second signal;
a frequency analyzer that analyzes a frequency component of the second signal;
a variance value calculator that calculates a first variance value of energy of at least one of the first frequency component and the second frequency component based on a result of analysis by the frequency analyzer;
a first statistical quantity calculator that calculates a first statistical quantity of the first variance value for a predetermined period; and
a determiner that determines presence or absence of a living body based on the first statistical quantity.

### Advantageous Effect of Invention

According to the disclosed technique, it is possible to accurately determine the presence or absence of a living body.

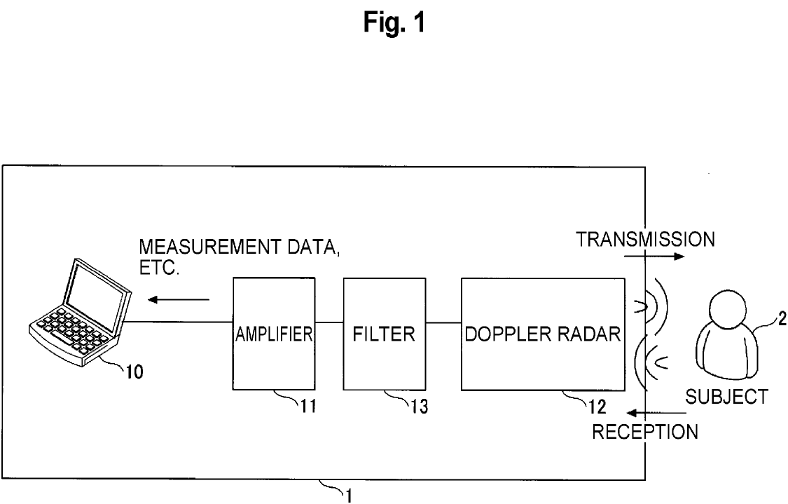

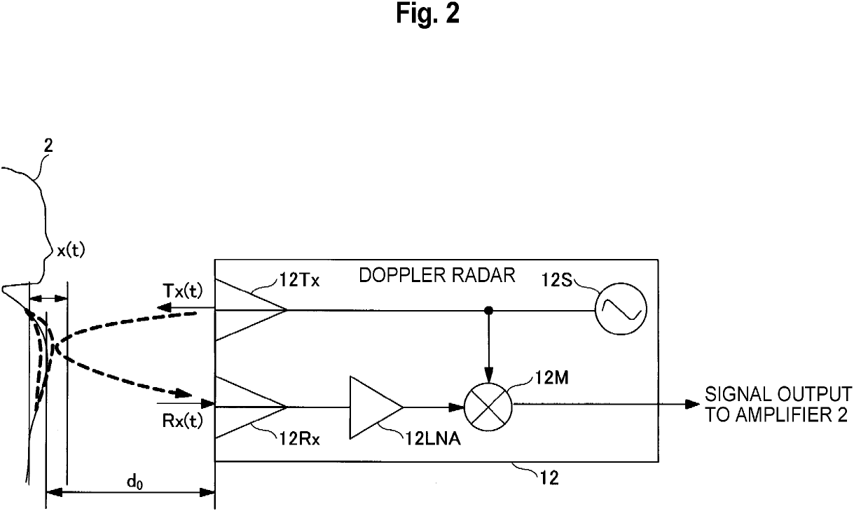

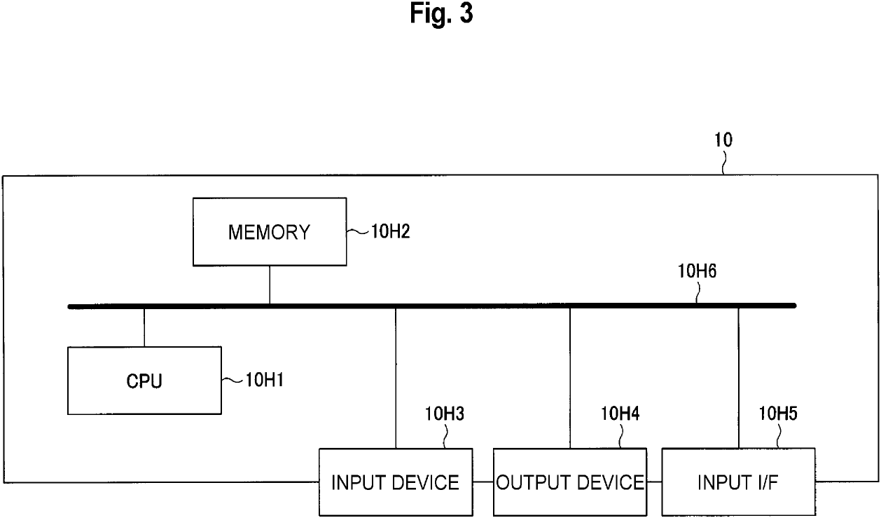

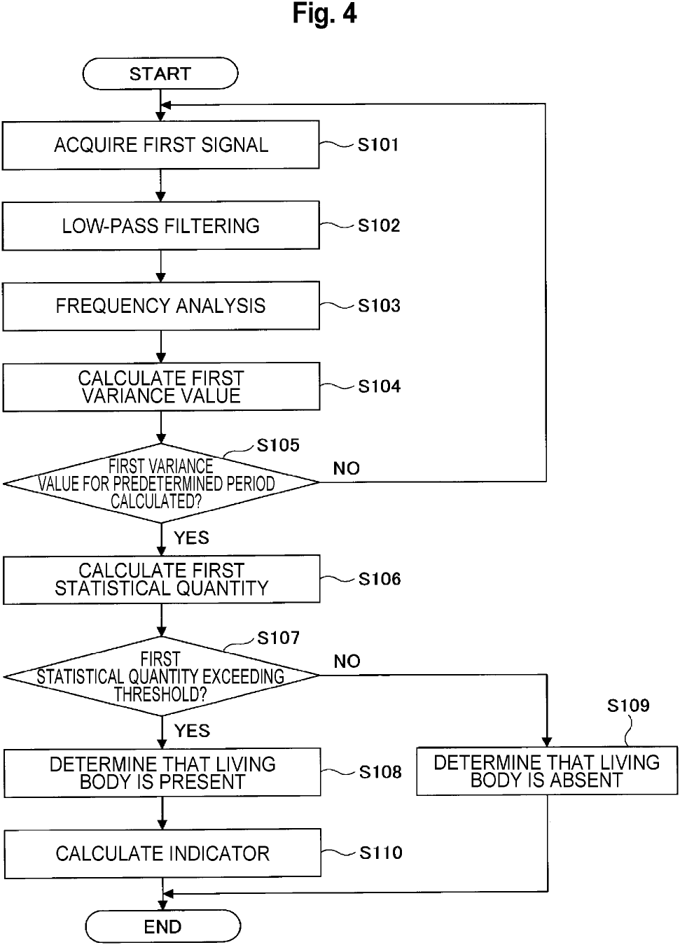

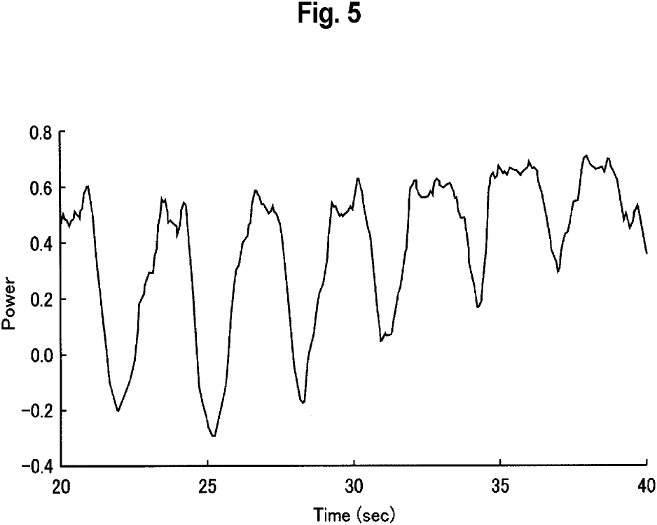

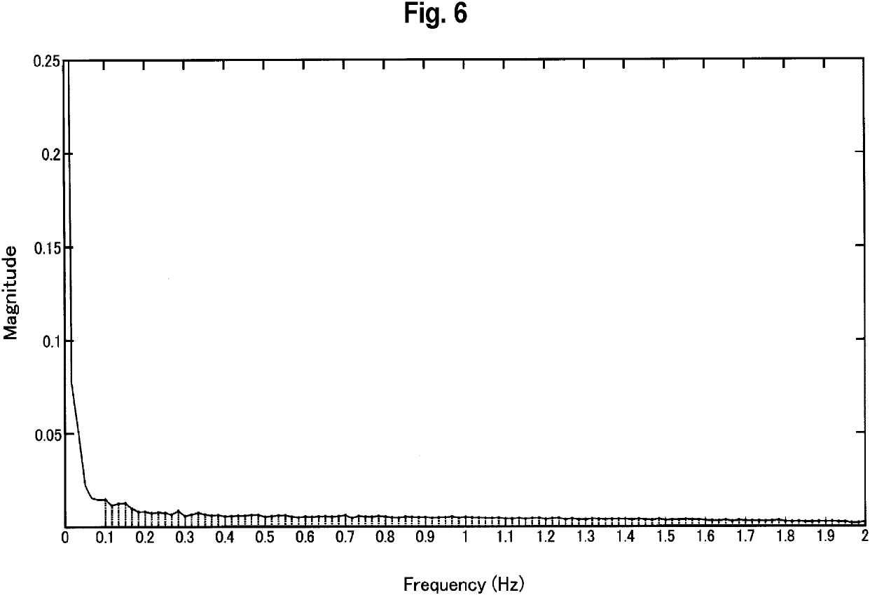

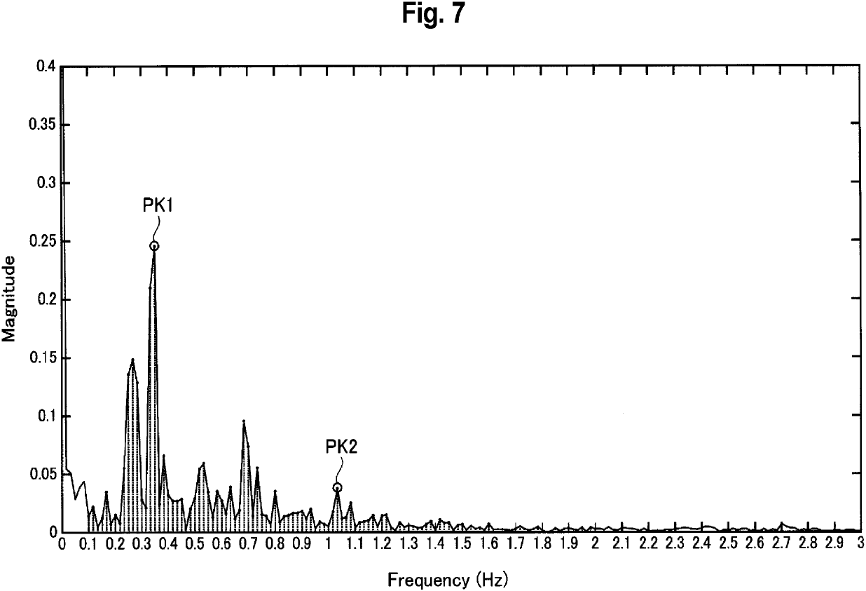

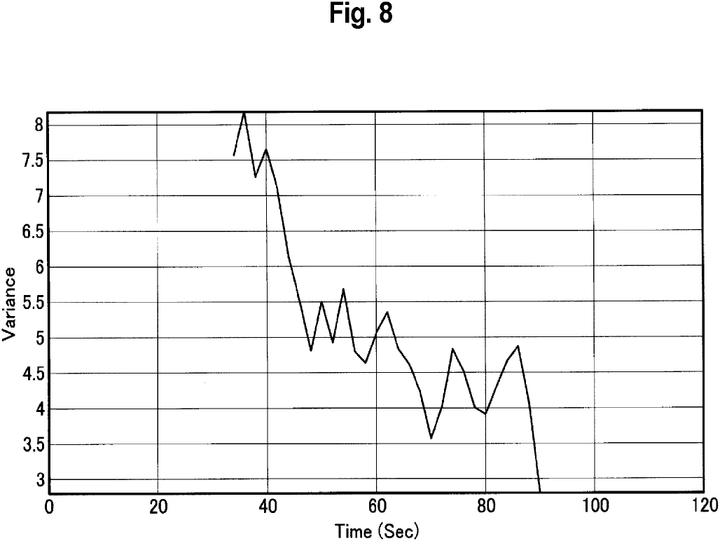

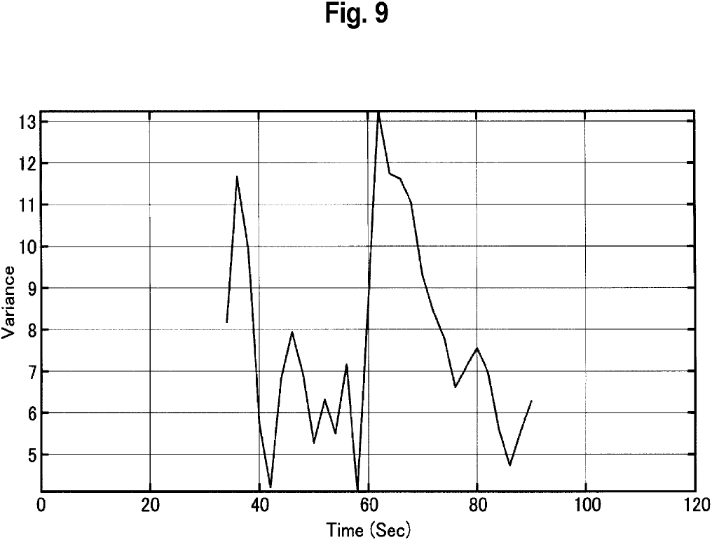

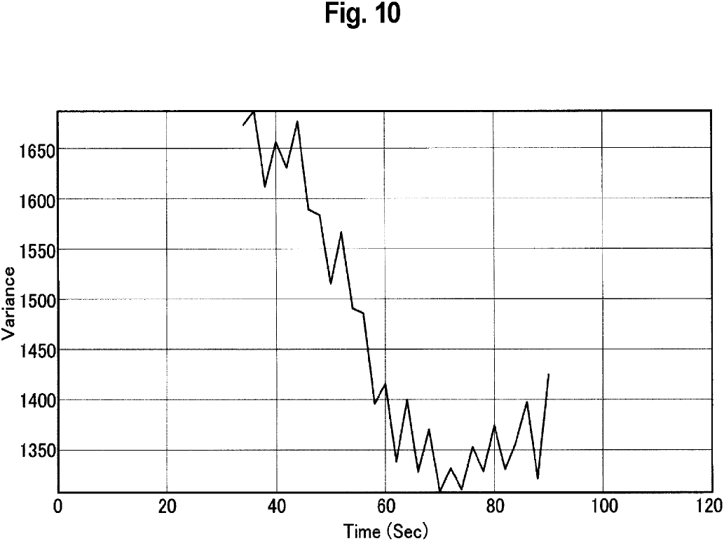

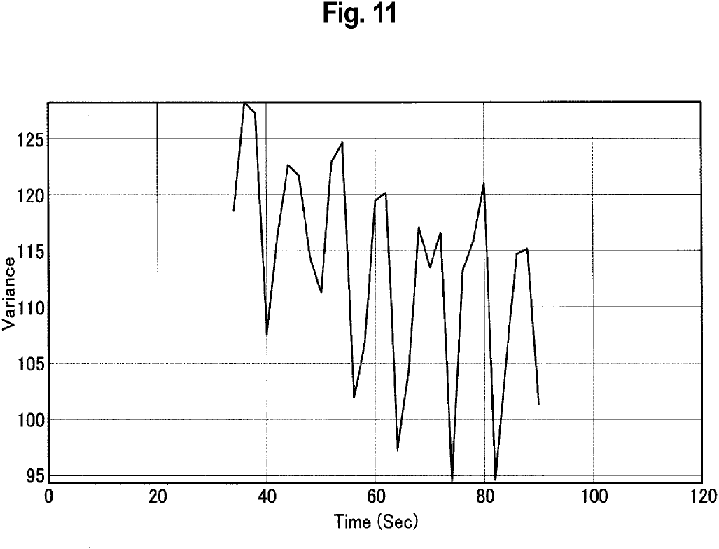

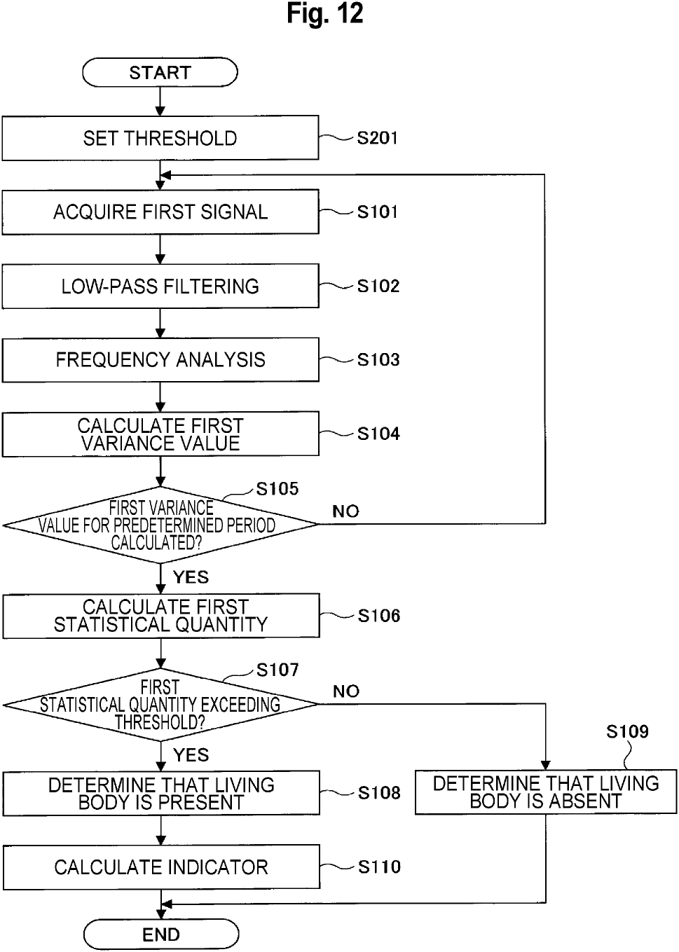

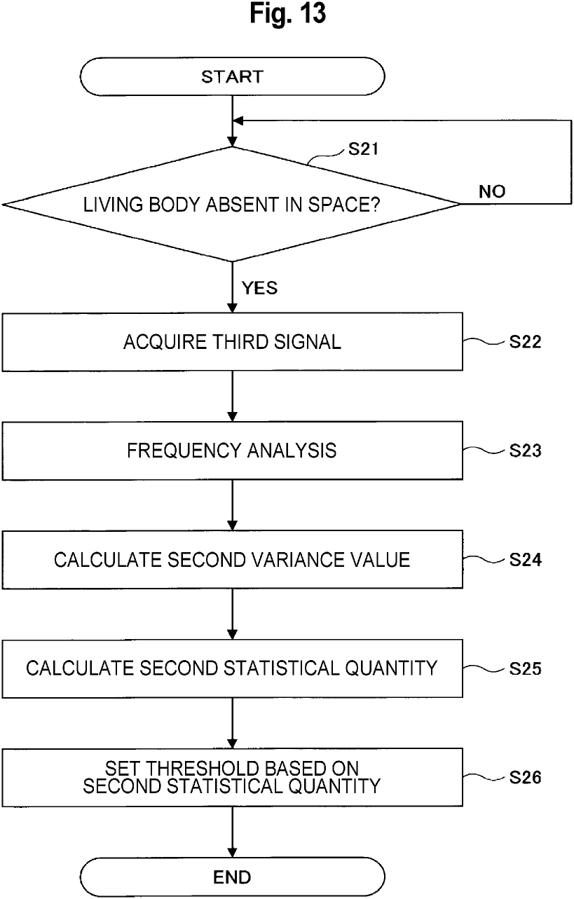

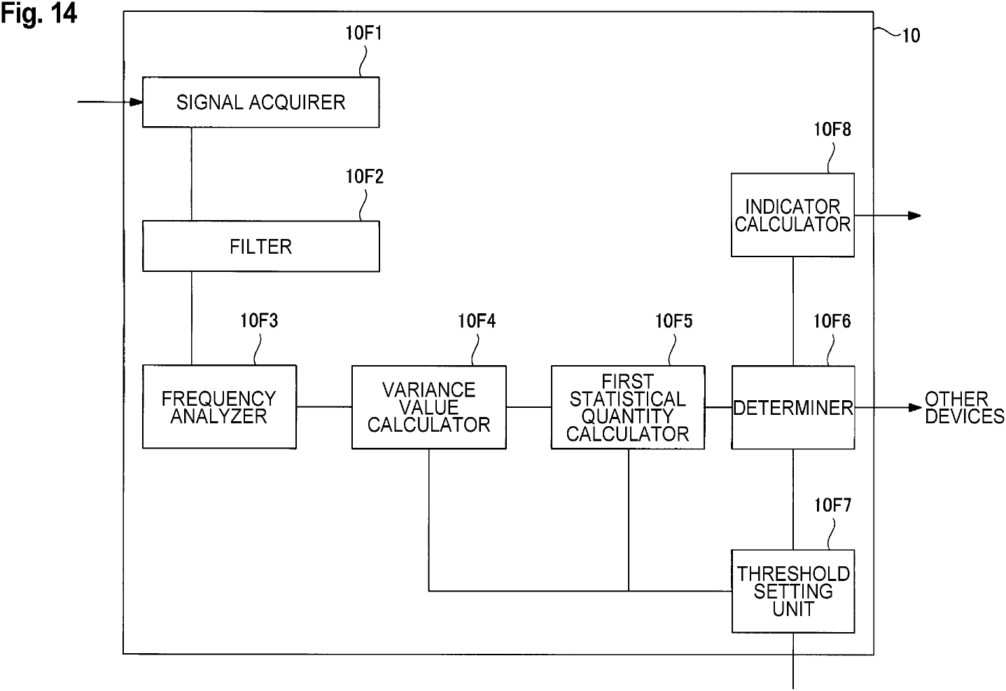

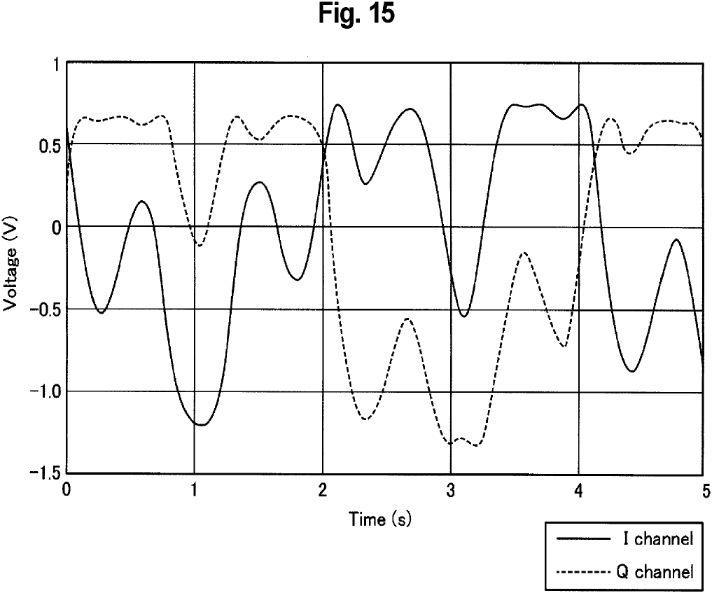

### BRIEF DESCRIPTION OF DRAWINGS

[FIG. 1] FIG. 1 shows an example overall configuration of a first embodiment.

[FIG. 2] FIG. 2 shows an example of a Doppler radar.

[FIG. 3] FIG. 3 shows an example of a living body detection device.

[FIG. 4] FIG. 4 shows an example overall process of the first embodiment.

[FIG. 5] FIG. 5 shows an example of a first signal.

[FIG. 6] FIG. 6 shows an analysis result in an experiment performed in the presence of a living body.

[FIG. 7] FIG. 7 shows an analysis result in an experiment performed in the absence of the living body.

[FIG. 8] FIG. 8 shows a first experimental result obtained by calculating a first variance value for a predetermined period in the absence of the living body.

[FIG. 9] FIG. 9 shows a second experimental result obtained by calculating the first variance value for the predetermined period in the absence of the living body.

[FIG. 10] FIG. 10 shows a first experimental result obtained by calculating the first variance value for the predetermined period in the presence of the living body.

[FIG. 11] FIG. 11 shows a second experimental result obtained by calculating the first variance value for the predetermined period in the presence of the living body.

[FIG. 12] FIG. 12 shows an example overall process of a second embodiment.

[FIG. 13] FIG. 13 shows an example of a process of setting a threshold.

[FIG. 14] FIG. 14 shows an example functional configuration.

[FIG. 15] FIG. 15 shows an example of IQ data measured by the Doppler radar.

### DESCRIPTION OF EMBODIMENTS

Optimal and minimal embodiments of the invention will be described below with reference to the drawings. Note that the same reference characters refer to similar components in the drawings, and overlapping descriptions will be omitted. Specific examples shown in the figures are illustrative, and further components other than those shown in the figures may also be included.

### First Embodiment

For example, a living body detection system 1 is a system with an overall configuration as described below.

### Example Overall Configuration

FIG. 1 shows an example overall configuration of a first embodiment. For example, the living body detection system 1 includes a personal computer (PC, hereinafter referred to as a “PC 10”), a Doppler radar 12, a filter 13 and the like. Note that the living body detection system 1 desirably includes an amplifier 11 or the like, as shown in the figure. The following description will be made with reference to the overall configuration shown in the figure by way of example.

The PC 10 is an information processing device and is an example of a living body detection device. The PC 10 is connected to peripheral devices such as the amplifier 11 via a network, a cable or the like. Note that the amplifier 11, the filter 13 and the like may be included in the PC 10. The amplifier 11, the filter 13 and the like may not be devices, but may be configured by software or configured by both hardware and software. The following description will be made with reference to the example of the living body detection system 1 as shown in the figure.

The Doppler radar 12 is an example of a measurement device.

In this example, the PC 10 is connected to the amplifier 11. The amplifier 11 is connected to the filter 13. The filter 13 is connected to the Doppler radar 12. The PC 10 acquires measurement data from the Doppler radar 12 via the amplifier 11 and the filter 13. That is, the measurement data is signal data indicating the action of a living body such as heartbeat and breathing. Next, the PC 10 analyzes the motion of a subject 2 such as heartbeat, breathing, and body movement based on the acquired measurement data, and measures the movement of the human body such as a heart rate.

The Doppler radar 12 acquires a signal (hereinafter referred to as a “biological signal”) indicating action such as heartbeat and breathing based on the following principle, for example.

### Example of Doppler Radar

FIG. 2 shows an example of the Doppler radar. For example, the Doppler radar 12 is a device with a configuration as shown in FIG. 2. Specifically, the Doppler radar 12 includes a source 12S, a transmitter 12Tx, a receiver 12Rx, and a mixer 12M. The Doppler radar 12 also includes an adjuster 12LNA such as a low noise amplifier (LNA) for performing a process such as reducing the noise in data received by the receiver 12Rx.

The source 12S is a transmission source for generating a transmission wave signal transmitted by the transmitter 12Tx.

The transmitter 12Tx transmits the transmission wave to the subject 2. Note that the transmission wave signal can be represented by a function Tx(t) with respect to time “t”, and can be represented as in equation (1) below, for example.

[Expression 1]

$$\text{Tx} \left(\text{t}\right) = \text{cos} \left({\text{ω}}_{\text{c}} \text{t}\right)$$

In equation (1) above, the letter “ _{c}” represents the angular frequency of the transmission wave.

It is assumed that the subject 2, that is, the reflection surface of the transmitted signal has a displacement of x(t) at time “t”. In this example, the reflection surface is the chest wall of the subject 2. The displacement x(t) can be represented as in equation (2) below, for example.

[Expression 2]

$$\text{x} \left(\text{t}\right) = \text{m} \times \text{cos} \left(\text{ω} \text{t}\right)$$

In equation (2) above, the letter “m” represents a constant indicating the amplitude of the displacement. Also, in equation (2) above, the letter “ω” represents the angular speed, which shifts due to the movement of the subject 2. Note that the variables similar to those in equation (1) above are the same variables.

The receiver 12Rx receives a reflected wave reflected by the subject 2 after being transmitted by the transmitter 12Tx. The reflected wave signal can be represented by a function Rx(t) with respect to time t, and can be represented as in equation (3) below, for example.

[Expression 3]

$$R x ( t ) = cos \left({ω}_{c} t - 2 π ⋅ \frac{2 \left({d}_{0} + x \left(t\right)\right)}{λ}\right)$$

In equation (3) above, the letter “d₀” represents the distance between the subject 2 and the Doppler radar 12. The letter “λ” represents the wavelength of the signal. The same notation applies hereinafter.

The Doppler radar 12 mixes the function Tx(t) (equation (1) above) indicating the transmission wave signal and the function R(t) (equation (3) above) indicating the reception wave signal to generate a Doppler signal. Note that the Doppler signal can be represented by a function B(t) with respect to time t, as in equation (4) below.

[Expression 4]

$$B \left(t\right) = cos \left(θ + 2 π ⋅ \frac{2 x ( t )}{λ}\right)$$

Defining the angular frequency of the Doppler signal as “ω_{d}”, the angular frequency ω_{d} of the Doppler signal can be represented as in equation (5) below.

[Expression 5]

$${ω}_{d} = θ + 2 π ⋅ \frac{2 x \left(t\right)}{λ}$$

The phase “θ” in equation (4) above and equation (5) above can be represented as in equation (6) below.

[Expression 6]

$$θ = 2 π ⋅ \frac{2 {d}_{0}}{λ} + {θ}_{0}$$

In equation (6) above, the letter “θ₀” represents the phase shift at the chest wall of the subject 2, that is, at the reflection surface.

Next, the Doppler radar 12 outputs the position, speed or the like of the subject 2 based on the result of comparing the transmitted transmission wave signal and the received reception wave signal, that is, the result of calculation in the equations above.

For example, I-data (in-phase data) and Q-data (quadrature-phase data) can be generated from the reception wave. Then, the distance by which the chest wall of the subject 2 moves can be detected by using the I-data and Q-data. It is also possible to detect whether the chest wall of the subject 2 moves frontward or backward based on the phase indicated by the I-data and Q-data. Therefore, the movement of the chest wall due to heartbeat can detect an indicator of the heartbeat or the like by using changes in the frequencies of the transmission wave and reception wave.

### Example Hardware Configuration of Living Body Detection Device

FIG. 3 shows an example of the living body detection device. For example, the PC 10 includes a central processing unit (CPU, hereinafter referred to as a “CPU 10H1”), a memory 10H2, an input device 10H3, an output device 10H4, and an input interface (I/F) (hereinafter referred to as an “input I/F 10H5”). Note that the hardware components included in the PC 10 are connected by a bus (hereinafter referred to as a “bus 10H6”), and data or the like is transmitted and received between the hardware components via the bus 10H6.

The CPU 10H1 is a control device for controlling the hardware components of the PC 10 and a computing device for performing computation for realizing various processing operations.

The memory 10H2 is a primary memory, an auxiliary memory and the like, for example. Specifically, the primary memory is a memory or the like, for example. The auxiliary memory is a hard disk or the like, for example. The memory 10H2 stores data including intermediate data used by the PC 10, programs used for various processing and control operations, and the like.

The input device 10H3 is a device for inputting parameters and instructions required for calculation to the PC 10 in response to an operation of the user. Specifically, the input device 10H3 is a keyboard, a mouse, a driver and the like, for example.

The output device 10H4 is a device for outputting various processing results and calculation results obtained by the PC 10 to the user or the like. Specifically, the output device 10H4 is a display or the like, for example.

The input I/F 10H5 is an interface connected to an external device such as a measurement device for transmitting and receiving data or the like. For example, the input I/F 10H5 is a connector, an antenna or the like. That is, the input I/F 10H5 transmits and receives data to/from the external device via a network, a wireless connection, a cable or the like.

Note that the hardware configuration is not limited to the configuration shown in the figure. For example, the PC 10 may further include a computing device, a memory or the like for performing processing in a parallel, distributed or redundant manner. The PC 10 may also be an information processing system connected to another device via a network or a cable for performing computation, control and storage in a parallel, distributed or redundant manner. That is, the present invention may be realized by an information processing system including one or more information processing devices.

The PC 10 thus acquires a biological signal indicating the action of the living body by using a measurement device such as the Doppler radar 12. Note that the biological signal may be acquired when necessary in real time, or may be collectively acquired by the PC 10 after a device such as the Doppler radar stores the biological signal for a certain period. A recording medium or the like may be used for the acquisition. The PC 10 may include a measurement device such as the Doppler radar 12, and the PC 10 may acquire the biological signal by performing measurement using the measurement device such as the Doppler radar 12 and generating the biological signal.

### Example Overall Process

FIG. 4 shows an example overall process. For example, the overall process described below is performed every time window (preset to 60 seconds, for example).

### Example of Acquiring First Signal

In step S101, the PC 10 acquires a first signal. For example, the first signal is a signal as shown below.

FIG. 5 shows an example of the first signal. In the figure, the horizontal axis indicates time, showing time points at which measurement is performed. The vertical axis indicates electric power estimated based on measurement results of the Doppler radar.

Hereinafter, a biological signal including a frequency component of heartbeat (hereinafter referred to as a “first frequency component”) and a frequency component of breathing (hereinafter referred to as second frequency component) as shown in the figure is referred to as a “first signal”.

### Example of Low Pass Filtering

In step S102, the PC 10 performs low-pass filtering on the first signal to attenuate frequency components higher than the first frequency component. That is, the PC 10 attenuates frequency components higher than the frequency component of heartbeat on the first signal. For example, the PC 10 performs filtering using a digital filter or the like with a cut-off frequency higher than the frequency component of heartbeat.

For example, since the heart rate of an adult male is about 50 to 180 beats per minute, the frequency component of heartbeat, that is, the first frequency component mainly contains frequency components of about 0.8 Hz to 3 Hz.

Also, for example, since the breathing rate of a person is about 10 to 60 breathes per minute, the frequency of breathing, that is, the second frequency component mainly contains frequency components of about 0.1 Hz to 1 Hz.

Thus, the low-pass filtering is desirably configured to attenuate frequency components higher than 0 Hz to 3 Hz, for example. With such configuration, the PC 10 can attenuate frequency components that would be noise without attenuating the frequency components indicating breathing and heartbeat through the low-pass filtering.

It is desirable that the low-pass filtering is performed not to attenuate frequency bands in which both breathing and heartbeat are included but to attenuate other frequency components higher than the first frequency component in which noise is included, as described above.

Note that the frequency bands targeted by the low-pass filtering may be set in consideration of the age, sex, state and the like of the living body. For example, in a state of having done a heavy exercise or a state of being agitated, both the heart rate and breathing rate have higher frequencies than in a resting state. Therefore, the first frequency component and the second frequency component are both at higher frequencies than in the resting state. On the other hand, in the resting state, both the heart rate and breathing rate are at low frequencies. Thus, the frequency bands targeted by the low-pass filtering may be dynamically changed or narrowed down according to a state or the like, for example. Specifically, in a state in which it is considered that both the first frequency component and the second frequency component are at a high frequency band, such as a state of having done a heavy exercise, low-pass filtering is performed to attenuate frequency components higher than 3.5 Hz. On the other hand, in a state in which it is considered that both the first frequency component and the second frequency component are at a low frequency band, such as a resting state, low-pass filtering is performed to attenuate frequency components higher than 1.4 Hz.

As described above, a state or the like can be input or a value may be set in consideration of a state or the like to perform the low-pass filtering.

For example, in the case of low-pass filtering set to 3 Hz, the PC 10 can attenuate frequency components of noise without attenuating the frequency components of heartbeat and breathing occurring after a heavy exercise that results in about 180 heartbeats per minute. Therefore, the low-pass filtering may be set to a low value of about 1 Hz such as in the case where it is known that the living body is not in a state of having done an exercise.

Hereinafter, a signal generated by the low-pass filtering is referred to as a “second signal”.

### Example of Frequency Analysis

In step S103, the PC 10 performs frequency analysis on the second signal. For example, the frequency analysis is realized by a fast Fourier transform (FFT) or the like. In this manner, the PC 10 calculates a spectrum indicating energy for each frequency band. It is desirable that the PC 10 indicates an analysis result in a normalized form and by a spectrum. Hereinafter, the spectrum is indicated by normalized values. A specific example of the analysis result will be described later.

### Example of Calculation of First Variance Value

In step S104, the PC 10 calculates a first variance value. The first variance value is a value indicating variance of energy in the analysis result of the frequency analysis of the second signal.

### Example of Determining Whether or Not First Variance Value for Predetermined Period is Calculated

In step S105, the PC 10 determines whether or not the first variance value for a predetermined period is calculated. For example, the predetermined period is two minutes. Note that the predetermined period is set in advance.

Next, if it is determined that the first variance value for the predetermined period is calculated (YES in step S105), the PC 10 proceeds to step S106. On the other hand, if it is determined that the first variance value for the predetermined period is not calculated (NO in step S105), the PC 10 proceeds to step S101. That is, steps S101 to S104 are repeatedly performed until the operations from the acquisition of the first signal to the calculation of the first variance value are finished for the predetermined period (which is two minutes in this example).

A specific example of the first variance value for the predetermined period will be described later.

### Example of Calculation of First Statistical Quantity

In step S106, the PC 10 calculates a first statistical quantity of the first variance value. That is, the PC 10 calculates, as the first statistical quantity, an average value of the first variance value for two minutes, in this example. Note that the first statistical quantity is a value calculated by performing statistical processing on a plurality of first variance values for the predetermined period, such as an average value, a variance value, a standard deviation or the like of the first variance value, for example. Further, the first statistical quantity may be a combination of two or more types of an average value, a variance value, or a standard deviation of the first variance value. That is, in step S107 later, two or more types of first statistical quantities may be used for the determination. The following description will be made with reference to an example in which the first statistical quantity is an average value of the first variance value.

### Example of Determining Whether or Not First Statistical Quantity Exceeds Threshold

In step S107, the PC 10 determines whether or not the first statistical quantity exceeds a threshold. The threshold is set in advance, for example. That is, the threshold for determining that the living body is present or determining that the living body is absent can be set by performing an experiment in advance, for example.

Note that the threshold is set according to the types and number of first statistical quantities. In this case, a different threshold may be set for each type of average value and variance value, or a single common threshold may be set and determination may be made separately.

Next, if it is determined that the first statistical quantity exceeds the threshold (YES in step S107), the PC 10 proceeds to step S108. On the other hand, if it is determined that the first statistical quantity does not exceed the threshold (NO in step S107), the PC 10 proceeds to step S109.

### Example of Determining that Living Body is Present

In step S108, the PC 10 determines that the living body is present.

### Example of Determining that Living Body is Absent

In step S109, the PC 10 determines that the living body is absent.

As in step S108 or step S109 shown above, the PC 10 determines the presence or absence of the living body based on the first statistical quantity. For example, in the case where the first statistical quantity is an average value of the first variance value for the predetermined period, if the average value of the first variance value is a high value, a determination result that the living body is present in a room or the like is output in step S108. On the other hand, if the average value of the first variance value is a low value, a determination result that the living body is absent in the room or the like is output in step S109.

Note that it is desirable that, when determining that the living body is present, the PC 10 performs calculation of an indicator or the like as described below.

### Example of Performing Calculation of Indicator

In step S110, the PC 10 calculates an indicator.

For example, the indicator is a value indicating biological information of the targeted living body. Specifically, the indicator is a value calculated by analyzing biological signals, and is a pulse rate, a heart rate, a breathing rate, a blood pressure, a pulse transit time (PTT), a systolic blood pressure, an R-R interval (RRI), a QRS interval, a QT interval, or a combination thereof, or the like. Note that the indicator may be other biological information.

The PC 10 may start from the acquisition of the biological signals in order to calculate the indicator.

As described above, in the living body detection system 1, the PC 10 calculates the indicator when determining that the living body is present. Such configuration avoids unnecessary measurement and processing, such as performing measurement even though the living body is absent. Thus, the calculation cost can be reduced, for example.

### Experimental Result

For example, the following analysis result is obtained as the analysis result of the frequency analysis, that is, step S103.

### Example of Analysis Result of Frequency Analysis

Hereinafter, a spectrum indicating frequency components on the horizontal axis and energy for each frequency component on the vertical axis is indicated by normalized values.

### Analysis Result in Absence of Living Body

FIG. 6 shows an analysis result in an experiment performed in the absence of the living body. The figure shows an analysis result of the frequency analysis in an experiment in which the first signal for one minute is acquired in the absence of the living body. In this experiment, the first variance value is “4.8 × 10⁻⁶”.

Thus, when the living body is absent, the first variance value is a low value.

### Analysis Result in Presence of Living Body

FIG. 7 shows an analysis result in an experiment performed in the presence of the living body. The figure shows an analysis result of the frequency analysis in an experiment in which the first signal for one minute is acquired in the presence of the living body. Note that the living body is a person lying on his/her back on a bed. The distance to the ceiling is “2.12 m”. In this experiment, the first variance value is “1426 × 10⁻⁶”.

Thus, when the living body is present, the first variance value is a high value.

When the living body is present, a peak point (a first peak point PK1 in the figure) appears in the frequency band of the second frequency component (a frequency band around 0.3 Hz in this example), which corresponds to the frequencies of breathing.

Similarly, when the living body is present, a peak point (a second peak point PK2 in the figure) appears in the frequency band of the first frequency component (a frequency band around 1 Hz in this example), which corresponds to the frequencies of heartbeat.

As described above, the frequency components indicating breathing and heartbeat have high energy in the second signal.

### Example of First Variance Value for Predetermined Period

The first variance value and the average value for the predetermined period obtained in the analysis result of the frequency analysis described above are as follows. Hereinafter, the horizontal axis indicates time, and the vertical axis indicates the first variance value. Note that the predetermined period is two minutes.

### First Variance Value in Absence of Living Body

FIG. 8 shows a first experimental result obtained by calculating the first variance value for the predetermined period in the absence of the living body.

FIG. 9 shows a second experimental result obtained by calculating the first variance value for the predetermined period in the absence of the living body.

FIGS. 8 and 9 show the transition of the first variance value over time. In the result shown in FIG. 8, the average value of the first variance value for two minutes is calculated as “5.2 × 10⁻⁶”. Further, in the result shown in FIG. 9, the average value of the first variance value for two minutes is calculated as “7.7 × 10⁻⁶” .

Thus, when the living body is absent, the average value of the first variance value for the predetermined period is a low value.

### First Variance Value in Presence of Living Body

FIG. 10 shows a first experimental result obtained by calculating the first variance value for the predetermined period in the presence of the living body.

FIG. 11 shows a second experimental result obtained by calculating the first variance value for the predetermined period in the presence of the living body.

FIGS. 10 and 11, like FIGS. 8 and 9, show the transition of the first variance value over time. In the result shown in FIG. 10, the average value of the first variance value for two minutes is calculated as “1457 × 10⁻⁶”. Further, in the result shown in FIG. 11, the average value of the first variance value for two minutes is calculated as “113.4 × 10⁻⁶”.

Thus, when the living body is present, the average value of the first variance value for the predetermined period is a high value.

Thus, the threshold used to determine the presence or absence of the living body is desirably a value by which the state as shown in FIGS. 8 and 9 and the state as shown in FIGS. 10 and 11 can be distinguished, for example.

Specifically, in the case of FIGS. 8 and 9 described above, that is, when the living body is absent, the average value of the first variance value for the predetermined period is a value less than or equal to “10 × 10⁻⁶”. On the other hand, in the case of FIGS. 10 and 11 described above, that is, when the living body is present, the average value of the first variance value for the predetermined period is a value greater than or equal to “100 × 10⁻⁶”. Thus, the threshold is desirably set to a value of about “14 × 10⁻⁶” to “20 × 10⁻⁶”, for example, in consideration of these experimental results. Such a threshold allows the PC 10 to accurately determine the presence or absence of the living body.

However, statistical quantities such as the first variance value and the average value vary greatly according to the normalization method, environment, living body and the like. Therefore, the threshold is not limited to the value shown in the above-described example, and is desirably set in consideration of these conditions.

In addition, the PC 10 determines the presence or absence of the living body by comparing at least one of an average value of the first variance value corresponding to the first frequency component and an average value of the first variance value corresponding to the second frequency component with the threshold. That is, the PC 10 may have an “OR” configuration in which it is determined that the living body is present if at least one of the average value of the first variance value corresponding to the first frequency component and the average value of the first variance value corresponding to the second frequency component is a high value. In other words, if the first variance value of the frequency component of either breathing or heartbeat is a high value, the PC 10 may determine that the living body is present even if the other first variance value is a low value. Thus, the PC 10 may make the determination by using either one of the first frequency component and the second frequency component.

However, the PC 10 desirably has an “AND” configuration in which it is determined on the whole that the living body is present if it is determined that the living body is present in both determinations for the average value of the first variance value corresponding to the first frequency component and the average value of the first variance value corresponding to the second frequency component.

That is, the PC 10 first calculates both the first variance value corresponding to the first frequency component and the first variance value corresponding to the second frequency component. Next, the PC 10 separately calculates the respective average values based on the respective first variance values. Then, the PC 10 determines that the living body is present when determining that the average value based on the first frequency component and the average value based on the second frequency component are both high values. Thus, the PC 10 is desirably configured to use the “AND” of both determinations for the first frequency component and the second frequency component. With such an “AND” configuration, the PC 10 can accurately determine the presence or absence of the living body.

### Second Embodiment

For example, the threshold used to determine the presence or absence of the living body may be set in the following process.

FIG. 12 shows an example overall process of a second embodiment. As compared to the first embodiment, the difference is that a process of setting the threshold is performed. Hereinafter, the difference from the first embodiment will be mainly described, and overlapping descriptions will be omitted.

### Example of Setting Threshold

In step S201, the PC 10 sets the threshold. Note that the type of the threshold is set in accordance with the type of the first statistical quantity calculated in step S106. Hereinafter, the case where the first statistical quantity calculated in step S106 is the average value of the first variance value for the predetermined period will be described by way of example. For example, the process of setting the threshold is the following process.

FIG. 13 shows an example of the process of setting the threshold.

### Example of Determining Whether or Not Living Body is Absent in Space

In step S21, the PC 10 determines whether or not the living body is absent in the space. That is, the PC 10 starts the process of setting the threshold after it is confirmed that the living body is absent in the space. Note that the determination of whether or not the living body is absent in the space may not be made by the PC 10 checking with a sensor or the like, but a result of determination by the user may be input for the determination.

Next, if it is determined that the living body is absent in the space (YES in step S21), the PC 10 proceeds to step S22. On the other hand, if it is determined that the living body is not absent in the space (NO in step S21), the PC 10 repeats step S21.

That is, a biological signal (hereinafter referred to as a “third signal”) generated in a space confirmed as a space in which the living body is absent is used to perform the subsequent processes.

### Example of Acquisition of Third Signal

In step S22, the PC 10 acquires the third signal. For example, the third signal is desirably generated by the Doppler radar, like the first signal.

### Example of Frequency Analysis

In step S23, the PC 10 performs frequency analysis on the third signal. For example, step S23 performs a process similar to step S103.

Note that the third signal may be subjected to low-pass filtering or the like before the frequency analysis. For example, in the case of an environment considered to contain much noise, even if the living body is absent in the space, the third signal may be subjected to low-pass filtering to attenuate the noise.

### Example of Calculation of Second Variance Value

In step S24, the PC 10 calculates a second variance value. For example, the second variance value is calculated in a manner similar to the first variance value, that is, in step S104. Hereinafter, a variance value used to set the threshold is referred to as a “second variance value”. Therefore, the second variance value is a value indicating variance of energy in the analysis result of the frequency analysis of the third signal.

### Example of Calculation of Second Statistical Quantity

In step S25, the PC 10 calculates a statistical quantity of the second variance value (a statistical quantity obtained by performing statistical processing on a plurality of second variance values is hereinafter referred to as a “second statistical quantity”). For example, like the first statistical quantity, an average value of the second variance value is calculated as the second statistical quantity and is set as a threshold in step S26 later.

### Example of Setting Threshold Based on Second Statistical Quantity

In step S26, the PC 10 sets a threshold based on the second statistical quantity. The PC 10 performs the determination of step S107 based on the threshold set in this manner.

Note that the threshold is not limited to the average value and may be another statistical quantity or the like obtained by using the second variance value calculated in step S24. Further, the threshold may also be a value based on the second statistical quantity such as a value obtained by adding a certain value to the average value calculated in step S25. Specifically, if the average value is a value of about “10 × 10⁻⁶” in step S25, the threshold may be set to a value of about “20 × 10⁻⁶” to “60 × 10⁻⁶” by adding a certain value of about “10 × 10⁻⁶” to “50 × 10⁻⁶” to “10 × 10⁻⁶”. Thus, in setting the threshold, some extent of allowance may be provided for the threshold. In addition, if a plurality of first statistical quantities are used, different thresholds used for respective determinations may be set.

Note that there is no need to perform the process of setting the threshold in succession as shown in the figure as long as the process is completed before performing the processes from step S101.

That is, there may be a time interval after performing the process of setting the threshold before performing the processes from step S101. In addition, the process of setting the threshold may be performed when a condition such as the targeted space or living body is changed, for example.

Thus, the PC 10 generates and acquires the third signal. The third signal is a biological signal indicating characteristics in a space in which the living body is absent, as it is generated in a space in which the living body is absent. The PC 10 desirably determines the presence or absence of the living body on the basis of the threshold set based on such a biological signal.

The threshold is desirably set differently in different environments of actual use, for example. Frequencies that appear in a space in which the living body is absent often differ in different environments. For example, the variance value in a space in which the living body is absent differs in an office or the like in which there is often a relatively small amount of noise or the like, in a factory or the like in which certain frequencies are often present characteristically, and in an ordinary home in which various frequencies are often present. Therefore, in order to set the threshold for each environment, the third signal is desirably acquired for each environment.

In the embodiments described above, it is possible to accurately determine the presence or absence of the living body.

### Third Embodiment

A third embodiment is a system including a living body detection device or a living body detection system (hereinafter referred to as a “system”). Note that the living body detection device and the living body detection system are similar to those in the first embodiment, and their descriptions will be omitted.

For example, the system starts the process or changes the process based on a result of determination of the presence or absence of the living body by the living body detection device or the living body detection system.

Specifically, in the system, the living body detection device or the living body detection system first determines the presence or absence of the living body in advance.

When determining that the living body is present, the living body detection device or the living body detection system starts other devices in the system. That is, since the living body detection device or the living body detection system determines that the living body is present such as when the living body enters a room, the living body detection device or the living body detection system performs a process of waking up the other devices or the like in synchronization with the entrance of the living body into the room. On the other hand, while it is determined that the living body is absent, the other devices or the like is in a sleeping mode, for example. If the living body detection device or the living body detection system starts other devices when determining that the living body is present as described above, there is no need for the devices or the like to perform unnecessary measurement, processing and the like. Thus, the power consumption can be reduced, for example. In addition, if the living body detection device or the living body detection system cooperates with a sensor or the like for detecting biological signals, false positives for the case where the living body is absent and false negatives can be reduced.

In addition, another device or the like may perform a process such as redetecting the living body or locating the living body when it is determined by the living body detection device or the living body detection system that the living body is present. Thus, the process of detecting the living body or generating the biological information may be started after the living body detection device or the living body detection system determines the presence or absence of the living body in advance and the presence of the living body is confirmed to some extent. If the detection of the living body is performed again by another device based on a result of determination by the living body detection device or the living body detection system as described above, it is possible to accurately detect the living body.

In addition, if the process of generating the biological information is started by another device based on a result of determination by the living body detection device or the living body detection system as described above, it is possible to accurately generate the biological information.

### Example Functional Configuration

FIG. 14 shows an example functional configuration. For example, the living body detection device desirably has a functional configuration including a signal acquirer 10F1, a filter 10F2, a frequency analyzer 10F3, a variance value calculator 10F4, a first statistical quantity calculator 10F5, and a determiner 10F6. In addition, the living body detection device desirably has a functional configuration further including a threshold setting unit 10F7 and an indicator calculator 10F8 as shown in the figure. The following description will be made with reference to the functional configuration as shown in the figure by way of example.

The signal acquirer 10F1 performs a signal acquisition procedure of acquiring a biological signal such as the first signal. For example, the signal acquirer 10F1 is realized by the Doppler radar 12, the input I/F 10H5 or the like.

The filter 10F2 performs a filter procedure of filtering a certain frequency band in the biological signal such as the first signal. For example, the filter 10F2 is realized by the CPU 10H1, the filter 13 or the like.

The frequency analyzer 10F3 performs a frequency analysis procedure of performing frequency analysis on a signal such as the second signal. For example, the frequency analyzer 10F3 is realized by the CPU 10H1 or the like.

The variance value calculator 10F4 performs a variance value calculation procedure of calculating a variance value such as the first variance value. For example, the variance value calculator 10F4 is realized by the CPU 10H1 or the like.

The first statistical quantity calculator 10F5 performs a first statistical quantity calculation procedure of calculating the first statistical quantity such as the average value of the first variance value. For example, the first statistical quantity calculator 10F5 is realized by the CPU 10H1 or the like.

The determiner 10F6 performs a determination procedure of determining the presence or absence of the living body. For example, the determiner 10F6 is realized by the CPU 10H1 or the like.

The threshold setting unit 10F7 performs a threshold setting procedure of setting the threshold. For example, the threshold setting unit 10F7 is realized by the CPU 10H1, the input device 10H3 or the like.

The indicator calculator 10F8 performs an indicator calculation procedure of calculating the indicator. For example, the indicator calculator 10F8 is realized by the CPU 10H1 or the like.

### Example of IQ Data Measured by Doppler Radar

FIG. 15 shows an example of IQ data measured by the Doppler radar. For example, the Doppler radar 12 outputs a signal as shown in the figure. The arctan (Q/I) is then calculated to obtain a biological signal.

The Doppler radar 12 can measure the movement of an object based on the Doppler effect, by which the frequency of reflected waves changes when a moving object is irradiated with radio waves. Such a configuration that can measure the movement of a subject in a contactless manner is desirable.

### Variation

Note that the living body is not limited to a human but may be an animal or the like.

The interval at which the biological signal is acquired and the predetermined period for which the first variance value is calculated may be set according to the application, accuracy and the like. For example, since the PC 10 can determine the presence or absence of the living body every short time if the predetermined period is about several seconds, the predetermined period or the like is desirably set to a short time of about several seconds in the case of outputting the presence or absence of the living body with a high time resolution.

On the other hand, regarding the first variance value, the average value and the determination, it is possible to accurately determine the presence or absence of the living body if the predetermined period is long. For example, if the predetermined period is set to a long time of four minutes or more, a large number of first variance values can be calculated, and therefore it is possible to more accurately determine the presence or absence of the living body.

The living body detection device and the living body detection system may be configured to use artificial intelligence (AI). For example, the threshold may be learned and set by machine learning or the like. Specifically, the machine learning is performed by using the variance value or the average value as in FIGS. 8 to 11 as training data. By using such training data, it is possible to generate a learned model that determines the presence or absence of the living body based on the variance value or the average value.

The living body detection device and the living body detection system may also perform deep learning by using a time domain signal or a frequency domain signal as a learning target. The living body detection device and the living body detection system may then determine the presence or absence of the living body based on the learned model.

The learned model is used as part of software in the AI. Therefore, the learned model is a program. Thus, the learned model may be distributed or executed via a recording medium, a network or the like, for example.

The learned model includes a network structure such as a convolution neural network (CNN) or a recurrent neural network (RNN), for example.

### Other Embodiments

For example, a transmitter, a receiver, or an information processing device may be a plurality of devices. That is, processing and control may be performed in a virtualized, parallel, distributed or redundant manner. On the other hand, the transmitter, receiver and information processing device may be integrated in hardware or share devices.

Note that all or part of each process according to the present invention may be written in a low-level language such as assembler or a high-level language such as an object-oriented language and realized by a program for causing a computer to perform the living body detection method. That is, the program is a computer program for causing a computer of the information processing device, the living body detection system or the like to perform each process.

Therefore, when each process is performed based on the program, a computing device and a control device included in the computer perform computation and control based on the program in order to perform each process. In order to perform each process, a memory included in the computer stores data used for the process based on the program.

The program can be recorded on a computer-readable recording medium and distributed. Note that the recording medium is a medium such as a magnetic tape, a flash memory, an optical disk, a magneto-optical disk or a magnetic disk. The program can be distributed through telecommunication lines.

Although preferred embodiments and the like have been described in detail above, there is no limitation to the above-described embodiments and the like, and various modifications and replacements can be made to the above-described embodiments and the like without departing from the scope of the claims.

This international application claims priority based on Japanese Pat. Application No. 2020-046621, filed on Mar. 17, 2020, the entire contents of which are hereby incorporated by reference into this international application.

### REFERENCE SIGNS LIST

1: living body detection system
2: subject
10F1: signal acquirer
10F2: filter
10F3: frequency analyzer
10F4: variance value calculator
10F5: first statistical quantity calculator
10F6: determiner
10F7: threshold setting unit
10F8: indicator calculator
11: amplifier
12: Doppler radar
13: filter
PK1: first peak point
PK2: second peak point

1. A living body detection device comprising:
a signal acquirer that acquires a first signal including a first frequency component that is a frequency component of heartbeat and a second frequency component that is a frequency component of breathing;
a filter that attenuates a frequency component higher than the first frequency component based on the first signal to generate a second signal;
a frequency analyzer that analyzes a frequency component of the second signal;
a variance value calculator that calculates a first variance value of energy of at least one of the first frequency component and the second frequency component based on a result of analysis by the frequency analyzer;
a first statistical quantity calculator that calculates a first statistical quantity of the first variance value for a predetermined period; and
a determiner that determines presence or absence of a living body based on the first statistical quantity.

2. The living body detection device according to claim 1, wherein the signal acquirer acquires the first signal by means of a Doppler radar.

3. The living body detection device according to claim 1 , wherein
the filter performs low-pass filtering to attenuate a frequency component higher than 3 Hz.

4. The living body detection device according to claim 1 , wherein
the first frequency component is a frequency component of 0.8 Hz to 3 Hz, and
the second frequency component is a frequency component of 0.1 Hz to 1 Hz.

5. The living body detection device according to claim 1 , wherein
the signal acquirer acquires a third signal that is a biological signal generated in a space in which the living body is absent,
the frequency analyzer analyzes a frequency component of the third signal,
the variance value calculator calculates a second variance value of energy based on a result of analysis of the frequency component of the third signal,
the determiner determines presence or absence of the living body on a basis of a threshold based on a second statistical quantity of the second variance value for a predetermined period, and
the living body detection device further comprises a threshold setting unit that sets the threshold.

6. The living body detection device according to claim 5, wherein the signal acquirer acquires the third signal by means of a Doppler radar.

7. The living body detection device according to claim 1 , wherein
the variance value calculator calculates respective first variance values for the first frequency component and the second frequency component,
the first statistical quantity calculator calculates respective first statistical quantities for the respective first variance values, and
the determiner determines that the living body is present if it is determined that the living body is present in both determinations based on the first statistical quantities.

8. The living body detection device according to claim 1 , further comprising:
an indicator calculator that calculates an indicator in relation to the living body if the determiner determines that the living body is present.

9. The living body detection device according to claim 1 , wherein
the first statistical quantity is an average value, a variance value, or a standard deviation of the first variance value, or a combination thereof.

10. A living body detection method performed by a living body detection device, the living body detection method comprising:
a signal acquisition procedure in which the living body detection device acquires a first signal including a first frequency component that is a frequency component of heartbeat and a second frequency component that is a frequency component of breathing;
a filter procedure in which the living body detection device attenuates a frequency component higher than the first frequency component based on the first signal to generate a second signal;
a frequency analysis procedure in which the living body detection device analyzes a frequency component of the second signal;
a variance value calculation procedure in which the living body detection device calculates a first variance value of energy of at least one of the first frequency component and the second frequency component based on a result of analysis by the frequency analysis procedure;
a first statistical quantity calculation procedure in which the living body detection device calculates a first statistical quantity of the first variance value for a predetermined period; and
a determination procedure in which the living body detection device determines presence or absence of a living body based on the first statistical quantity.

11. A program for causing a computer to perform the living body detection method according to claim 10.
